(19) United States
(12) Patent Application Publication
(10) Pub. No.: US 2023/0079850 A1
(43) Pub. Date: Mar. 16, 2023

(54) METHODS OF MAKING NON-WOVEN MATERIALS FROM MYCELIUM

(71) Applicant: TEKNOLOGIAN TUTKIMUSKESKUS VTT OY, Espoo (FI)

(72) Inventors: Geza SZILVAY, Espoo (FI); Christiane LAINE, Espoo (FI); Manuel ARIAS BARRANTES, Espoo (FI); Anniina SUHONEN, Espoo (FI); Harry BOER, Espoo (FI); Merja PENTTILÄ, Espoo (FI); Pauliina AHOKAS, Espoo (FI)

(21) Appl. No.: 17/790,186

(22) PCT Filed: Dec. 30, 2020
(86) PCT No.: PCT/FI2020/050875

§ 371 Date: Jun. 30, 2022

(30) Foreign Application Priority Data
Dec. 31, 2019 (FI) 20196139

(51) Int. Cl. C08L 1/02; A01G 18/20; C12N 1/14; C12P 1/02; D06N 3/02

(52) U.S. Cl. CPC C08L 1/02; A01G 18/20; C12N 1/14; C12P 1/02; D06N 3/02; C12R 2001/645

(57) ABSTRACT

The present invention relates to a method of making non-woven material from mycelium produced in a stirred submerged liquid culture. The present invention also relates to use of a crosslinking agent in making a non-woven material from mycelium produced in a stirred submerged liquid culture. In addition, the present invention relates to use of mixing in making a non-woven material from mycelium produced in a stirred submerged liquid culture. The present invention relates also to a mycelium based non-woven material, wherein the mycelium is produced in a stirred submerged liquid culture.

### TECHNICAL FIELD

The present invention relates to a method of making non-woven material from mycelium produced in a stirred submerged liquid culture. The present invention also relates to use of a crosslinking agent in making a non-woven material from mycelium produced in a stirred submerged liquid culture. In addition, the present invention relates to use of agitation in making a non-woven material from mycelium produced in a stirred submerged liquid culture. The present invention relates also to a mycelium based non-woven material, wherein the mycelium is produced in a stirred submerged liquid culture.

### BACKGROUND

Market for leather goods is 600 B$ and the CAGR is estimated at 6%. However, leather production is problematic due to several reasons including: i) effect of toxic tanning chemicals to workers and environment, ii) significant GHG emissions from meat production, iii) land use problems such as deforestation. Therefore, there is an increasing interest in leather alternatives. New approaches include bio-based materials such as pineapple leave fibres and fungal mycelium. Fungal mycelium can be used to make flexible leather-like sheets or fabrics. In fact, in Europe mushrooms have been used to make hats and belts for hundreds of years. Mycelium consists of a highly branched micro-sized network of growing filamentous cells. The mushroom consists of the same cells but which have differentiated to a simple tissue-like structure.

The current industrial process for producing mycelium materials is growing the mycelium in a static solid-state fermentation for 2-3 weeks. An alternative method is to culture mushrooms and process them into fabrics. Document WO 2018/183735 discloses solution based post-processing methods for mycological biopolymer material and mycological product made thereby. Document US 2015/0038326 discloses chemically modified mycological materials having absorbent properties.

Current methods for producing mycelium materials are slow, poorly controlled, difficult to scale up, require specialized growth chambers, and the facilities require a large footprint. These problems may increase the production costs and hinder the products from entering the market.

### BRIEF DESCRIPTION OF THE INVENTION

An object of the present invention is to provide a method of making non-woven material from mycelium produced in a stirred submerged liquid culture, wherein the method comprises treating the mycelium suspension with a crosslinking agent and/or agitating the mycelium suspension during the preparation process. Another object of the present invention is to provide a method of making non-woven material from mycelium produced in a stirred submerged liquid culture, wherein the method comprises treating the mycelium suspension with a crosslinking agent. A further object of the present invention is to provide a method of making non-woven material from mycelium produced in a stirred submerged liquid culture, wherein the method comprises agitating the mycelium suspension during the preparation process.

An object of the present invention is also a method of making non-woven material from mycelium, wherein the method comprises producing the mycelium in a stirred submerged liquid culture, treating the mycelium with a crosslinking agent and/or agitating the mycelium suspension during the preparation process.

In addition, the present invention relates to use of a crosslinking agent in making a non-woven material from unmodified mycelium produced in a stirred submerged liquid culture. In addition, the present invention relates to use of agitation in making a non-woven material from mycelium produced in a stirred submerged liquid culture. A further object of the present invention is to provide a method of making non-woven material from mycelium in a stirred submerged liquid culture. Further, an object of the present invention is to provide mycelium based non-woven material wherein the mycelium is produced in a stirred submerged liquid culture.

In the present invention, a chemical process that strengthens the mycelium material's physical properties has been developed. In addition, in the present invention a mechanical process that strengthens the mycelium material's physical properties has been developed. The processes are water based and use mainly environmentally friendly chemicals. The processes enable mycelium produced in a stirred submerged liquid culture to be processed into a non-woven textile-like material. Moreover, the chemical process may also be used to strengthen the materials produced by the typical solid state fermentation method.

The objects of the invention are achieved by the product, the method and the use characterized by what is stated in the independent claims. The preferred embodiments of the invention are disclosed in the dependent claims.

### BRIEF DESCRIPTION OF THE DRAWINGS

FIG. 1 shows flow diagrams of exemplary mycelium non-woven material preparation processes A-E.

FIG. 2 shows the cross-linked mycelium non-woven materials of Ganoderma lucidum, Pleurotus ostreatus, Fomes fomentarius and Trichoderma reesei.

FIG. 3 shows the effect of citric acid cross-linking on improving the tensile strength of non-woven mycelium material. Data shown as relative to control samples.

FIG. 4 shows the tensile strength (relative to the control sample) and percentage strain of non-woven mycelium material before and after glutaraldehyde cross-linking.

FIG. 5 shows the effect of cellulose pulp on tensile strength (relative to the control sample) and percentage strain of non-woven mycelium material.

FIG. 6 shows the tannin and enzyme treated mycelium non-woven materials. Tannin containing samples have a leathery feel.

FIG. 7 shows the tensile strength (relative to the control sample) and percentage strain of non-woven mycelium material after tannin treatment.

FIG. 8 shows the tensile strength (relative to the control sample) and percentage strain of non-woven mycelium materials without and with stirring.

FIG. 9 shows the change in tensile strength of non-woven mycelium material with increasing nanocellulose fibril content (relative to the control sample).

### DETAILED DESCRIPTION OF THE INVENTION

The present invention is based on a finding that non-woven material can be produced from mycelium cultivated in a stirred submerged liquid culture. Producing sheets of non-woven material from submerged liquid cultures is very different from producing such material from mycelium cultured in a solid state fermentation process as the mycelium microstructures are different from each other. Using stirred submerged liquid cultures is relatively inexpensive, fast, scalable, on-line controllable method. In addition, readily available bioreactors can be used. Moreover, the production method allows the controlling of mycelium interaction with itself and with added fibres. The present invention shows that component interaction within unmodified mycelium non-woven material can be increased by carefully controlling the colloidal state of the mycelium, by cross-linking the mycelium, and/or by incorporating reinforcing fibres. The colloidal state of the mycelium can be controlled by agitating the mycelium, for example. Cross-linking and incorporation of fibres have been shown before for mycelium sheets from solid state fermentation and for modified (deacetylated) mycelium. In the present invention, however, it was found that mycelium non-woven materials can be formed from unmodified mycelium produced in a stirred submerged liquid cultivation by controlling the mycelium colloidal state, cross-linking and/or incorporating of reinforcing fibres.

The present invention thus relates to a method of making non-woven material from mycelium produced in a stirred submerged liquid culture wherein the method comprises treating the mycelium suspension with a crosslinking agent and/or agitating the mycelium suspension during the preparation process of the non-woven material. In one embodiment, the invention relates to a method of making non-woven material from mycelium produced in a stirred submerged liquid culture, wherein the method comprises treating the mycelium suspension with a crosslinking agent during the preparation process of the non-woven material. In one embodiment, the invention relates to a method of making non-woven material from mycelium produced in a stirred submerged liquid culture, wherein the method comprises treating the mycelium with agitation and/or mixing during the preparation process of the non-woven material. In one embodiment, the present invention relates to use of a cross-linking agent in making a non-woven material from unmodified mycelium, which is produced in a stirred submerged liquid culture. In a further embodiment, the invention relates to use of agitation for controlling the colloidal state of the mycelium in making a non-woven material from mycelium which is produced in a stirred submerged liquid culture. The invention also relates to a method of making a non-woven material from mycelium in a stirred bioreactor. In addition, the invention relates to non-woven material based on mycelium produced in a stirred submerged liquid culture.

In the method of the present invention cross-linking of fungal mycelium in liquid suspension is performed without an impregnation or soaking process. The cross-linking of the mycelium takes place during the film formation or during the curing step. Further, in the method of the present invention there is no need to expose the submerged liquid culture to a subsequent static cultivation or growing step to induce cohesion between mycelium for a material.

In the present invention, the mycelium is cultured/cultivated in stirred/mixed liquid suspension. Accordingly, in the present invention the mycelium is produced in stirred submerged liquid culture or cultivation conditions. In one embodiment, the mycelium is produced by stirred liquid fermentation. In one embodiment, the mycelium is produced in stirred liquid bioreactor cultivation. In one embodiment, the mycelium is cultivated in bubble column reactor. In one embodiment, the mycelium is produced in shake flask cultivation. In one embodiment, the mycelium is produced by culturing in stirred liquid broth. In one embodiment, the mycelium is submerged in stirred liquid with contact to nutrients in soluble or insoluble form. In one embodiment, the mycelium is produced in stirred semi-solid submerged liquid cultivation. Equipments suitable for producing mycelium in stirred submerged liquid culture or cultivation conditions are known by the skilled person and include stirred bioreactors and bubble column reactors, for example. As used herein the term “semi-solid” refers to a liquid broth, which contains ground organic material, such as ground vegetable peels and/or sawdust, for example. In the present invention, solid state fermentation or static liquid cultivation is not exploited in the cultivation and/or production of the mycelium. As used herein the term “stirred/stirring” refers also to mixed/mixing and/or bubbled/bubbling. In the present invention, the stirring can be performed by any conventional means known to a skilled person, such as stirring, mixing and/or bubbling.

In the present invention, the cell-walls of mycelium can be chemically cross-linked. Addition of a cross-linking agent, such as citric acid, was found to increase the strength and stiffness of the mycelium material while preserving its extendibility. Without wishing to be bound by a theory, the crosslinking agent reacts with the cell wall glucan hydroxyl groups and cross-links the cell walls of neighbouring filaments together.

In addition, agitation of the mycelium culture during the preparation process of the non-woven material was found to increase the strength and stiffness of the mycelium material while preserving its extendibility. Mixing and/or agitation opens up the entangled mycelium structure, dispersing the mycelium, and therefore enhancing interactions within the final material. The mixing and/or agitation likely also induces changes in the fungal metabolism and cell wall structure. The mixing needs to be vigorous while avoiding break down of the mycelium.

In one embodiment, the crosslinking agent is a polycarboxylic acid. In one embodiment, the crosslinking agent is a tricarboxylic acid. In one embodiment, the crosslinking agent is a tricarboxylic acid selected from citric acid/or succinid acid. In one embodiment, the crosslinking agent is a dicarboxylic acid. In one embodiment, the crosslinking agent is a dicarboxylic acid selected from galactaric acid and/or suberic acid. In one embodiment, the crosslinking agent is glutaraldehyde. In one embodiment, the crosslinking agent is a tannin. In one embodiment, an enzyme, such as oxidase or an oxidoreductase is used to crosslink tannins, lignin or vanillin into the mycelium material. In one embodiment, laccase or tyrosinase is used to crosslink tannins, lignin or vanillin into the mycelium material.

In one embodiment, the crosslinking agent is separately added to the mycelium.

In one embodiment, the crosslinking is performed with a heat-treatment. In one embodiment, the heat-treatment is performed at a temperature ranging from about 90° C. to about 150° C. In one embodiment, the heat-treatment is performed at a temperature ranging from about 100° C. to about 120° C. In one embodiment, the heat-treatment is performed for a time period of about 0.5 min to 60 min. In one embodiment, the heat-treatment is performed for a time period of about 1 min to about 10 min. In one embodiment, the heat-treatment is performed at a temperature ranging from about 90° C. to about 150° C. for a time period of about 0.5 min to 60 min. In one embodiment, the heat-treatment is performed at a temperature ranging from about 90° C. to about 150° C. for a time period of about 1 min to about 10 min. In one embodiment, the heat-treatment is performed at a temperature ranging from about 100° C. to about 120° C. for a time period of about 0.5 min to 60 min. In one embodiment, the heat-treatment is performed at a temperature ranging from about 100° C. to about 120° C. for a time period of about 1 min to about 10 min.

In one embodiment, a fungal strain producing the crosslinking agent, such as a dicarboxylic or a tricarboxylic acid, can be used without the need to add the cross-linking agent as a chemical. Accordingly, in one embodiment, the crosslinking agent is produced by the fungus producing the mycelium.

In one embodiment, a plasticizer or softener is used together with the cross-linking agent. The plasticizer can be selected from glycols, sugar alcohols, epoxy esters, ester plasticizers, glycerol esters, phosphate esters, terephtalates, leather conditioners, acetylated monoglycerides, alkyl citrates, epoxidized vegetable oils, methyl ricinoleate, other common polymer plasticizers or any mixture thereof. In one embodiment, the plasticizer is a glycol, such as propylene glycol, polyethylene glycol or a mixture thereof. In one embodiment, the plasticizer is a sugar alcohol, such as glycerol, sorbitol, xylitol or a mixture thereof. In one embodiment, the plasticizer is a leather conditioner, such as a wax, an oil or a mixture thereof. In one embodiment, the plasticizer is a mixture of a sugar alcohol and a glycol.

In one embodiment, mycelium is treated with mixing and/or agitation in and/or during the preparation process. In one embodiment, a plasticizer or softener is used together with the mixing and/or agitation. In one embodiment, the plasticizer is a sugar alcohol. In one embodiment, the plasticizer is glycerol and/or sorbitol and/or xylitol. In one embodiment, the plasticizer is polyethylene glycol. In one embodiment, the mycelium is mixed for a period of time from 5 min to 12 hours. In one embodiment, the mycelium is mixed for a period of time from 5 min to 1 hour. In one embodiment, the mycelium is mixed for about 30 min.

In one embodiment, the mycelium is combined with a polymer, a fiber and/or a colouring agent during the cultivation. In one embodiment, the mycelium is combined with a polymer, a fiber and/or a colouring agent after the cultivation. This enables the formation of different kinds of blends and/or composites of mycelium together with polymers and/or polysaccharides and/or colouring agents. Suitable fibers and/or polymers include nanocellulose fibrils, cellulose nanofibrils, nanofibrillated cellulose and cellulose pulp, for example. Suitable polymers include cellulose derivatives and/or polyvinyl alcohol, for example.

The method of the present invention can comprise also additional steps such as casting and/or curing, for example. Thus, in one embodiment, the method of making the mycelium-based non-woven material of the present invention comprises also a step of casting. In one embodiment, the method of making the mycelium-based non-woven material of the present invention comprises also a step of curing.

In one embodiment, the mycelium is produced by stirred bioreactor cultivation. The invention enables the production of mycelium materials using stirred bioreactor cultivations. The benefits of using stirred submerged liquid cultivation, such as stirred bioreactors, as compared to solid-state fermentation trays include faster growth (5 days vs. 20-30 days), the production is easier to scale, the production is online controllable, and no special facilities are needed as culturing can be done in normal bioreactors. The used chemicals are mainly environmental friendly and safe. For example citric acid is frequently used in foods. Tannins and lignin are also bio-based additives.

In one embodiment, the mycelium is derived from a Trichoderma reesei strain. In one embodiment, the mycelium is derived from a Ganoderma lucidum strain. In one embodiment, the mycelium is derived from a Pleurotus ostreatus strain. In one embodiment, the mycelium is derived from a Fomes fomentarius strain.

The present invention relates also to a method of making non-woven material from mycelium in a stirred submerged liquid cultivation, wherein the method comprises the steps of:
a) providing a mycelium pre-culture, a nutrient and means for stirred submerged liquid cultivation conditions,
b) culturing the mycelium in the stirred submerged liquid cultivation,
c) separating the mycelium from the nutrient media,
d) optionally washing the mycelium with water,
e) optionally adding a crosslinking agent,
f) optionally mixing the mycelium,
g) adding a plasticizer to the mycelium suspension in one of the steps b) to f) or to the mycelium after the step h),
h) drying the mycelium,
i) optionally heat-treating the mycelium.

In one embodiment, the method of making non-woven material from mycelium in a stirred submerged liquid cultivation comprises the steps of:
a) providing a mycelium pre-culture, a nutrient and means for stirred submerged liquid cultivation,
b) culturing the mycelium in the a stirred submerged liquid cultivation conditions,
c) separating the mycelium from the nutrient media,
d) optionally washing the mycelium with water,
e) adding a crosslinking agent,
f) adding a plasticizer to the mycelium suspension in one of the steps b) to e) or to the mycelium after the step g),
g) drying the mycelium,
h) optionally heat-treating the mycelium.

In one embodiment, the method of making non-woven material from mycelium in a stirred submerged liquid cultivation comprises the steps of:
a) providing a mycelium pre-culture, a nutrient and a plasticizer and means for stirred submerged liquid cultivation,
b) culturing the mycelium with plasticizer in the stirred submerged liquid cultivation conditions,
c) separating the mycelium from the nutrient media,
d) optionally washing the mycelium with water,
e) optionally adding a crosslinking agent,
f) drying the mycelium
g) optionally heat-treating the mycelium.

In one embodiment, the method of making non-woven material from mycelium in a stirred submerged liquid cultivation comprises the steps of:
a) providing a mycelium pre-culture and a nutrient and means for stirred submerged liquid cultivation,
b) culturing the mycelium in the stirred submerged liquid cultivation condition,
c) separating the mycelium from the nutrient media,
d) optionally washing the mycelium with water,
e) adding a plasticizer to the mycelium suspension in one of the steps b) to e) or to the mycelium after the step g),
f) mixing the mycelium,
g) drying the mycelium.

In one embodiment, the method of making non-woven material from mycelium in a stirred submerged liquid cultivation comprises the steps of:
a) providing a mycelium pre-culture and a nutrient and means for stirred submerged liquid cultivation,
b) culturing the mycelium in the stirred submerged liquid cultivation conditions,
c) separating the mycelium from the nutrient media,
d) optionally washing the mycelium with water,
e) adding a plasticizer to the mycelium suspension in one of the steps b) to d) or to the mycelium after the step g),
f) mixing the mycelium,
g) drying the mycelium.

In one embodiment, the method of making non-woven material from mycelium in a stirred submerged liquid cultivation comprises the steps of:
a) providing a mycelium pre-culture, a nutrient, a plasticizer and means for stirred submerged liquid cultivation,
b) culturing the mycelium with plasticizer, in the stirred submerged liquid cultivation conditions,
c) separating the mycelium from the nutrient media,
d) washing the mycelium with water,
e) mixing the mycelium,
f) drying the mycelium.

In the method of the present invention, the crosslinking agent such as a dicarboxylic or a tricarboxylic acid, can be produced by the fungus producing the mycelium. Thus, there is no need to add the cross-linking agent as a chemical. Accordingly, in one embodiment, the method of making non-woven material from mycelium in a stirred submerged liquid cultivation comprises the steps of:
a) providing a mycelium pre-culture, a nutrient and means for stirred submerged liquid cultivation,
b) culturing the mycelium in the stirred submerged liquid cultivation conditions,
c) separating the mycelium from the nutrient media,
d) optionally washing the mycelium with water,
e) adding a plasticizer to the mycelium suspension in one of the steps b) to d) or to the mycelium after the step f),
f) drying the mycelium
g) optionally heat-treating the mycelium.

In another embodiment, the method of making non-woven material from mycelium in a stirred submerged liquid cultivation comprises the steps of:
a) providing a mycelium pre-culture, a nutrient, a plasticizer and means for stirred submerged liquid cultivation,
b) culturing the mycelium with plasticizer in the stirred submerged liquid cultivation conditions,
c) separating the mycelium from the nutrient media,
d) washing the mycelium with water,
e) drying the mycelium,
f) optionally heat-treating the mycelium.

In the method of the present invention, drying of the mycelium is performed with any technique known by a skilled person. Thus, in one embodiment of the invention, the mycelium is dried in a solvent casting, a film casting, a wet-laying or a paper-making process. Drying of the mycelium is however not limited to these processes in the present invention. The drying can be performed at room temperature or at a temperature ranging from about 60° C.-100° C. or about 70° C.-90° C., for example.

The method of the present invention can comprise also additional steps such as adding fibers and/or polymers, adding colouring agents, casting and/or curing, for example. The fibers and/or polymers can be used to reinforce the structure of the material, for example. The method of the present invention can comprise also an additional step of adding an enzyme, such as oxidase or an oxidoreductase. Thus, in one embodiment, the method of making the mycelium-based non-woven material of the present invention comprises also a step of adding fibers and/or polymers. In one embodiment, the method of making the mycelium-based non-woven material of the present invention comprises also a step of adding a colouring agent. In one embodiment, the method of making the mycelium-based non-woven material of the present invention comprises also a step of adding an enzyme. In one embodiment, the method of making the mycelium-based non-woven material of the present invention comprises also a step of casting. In one embodiment, the method of making the mycelium-based non-woven material of the present invention comprises also a step of curing.

In one embodiment, the method of making the mycelium-based non-woven material of the present invention consists essentially of the steps a) to f), g), h) or i), respectfully.

In one embodiment, the mycelium is derived from a Trichoderma reesei strain. In one embodiment, the mycelium is derived from a Ganoderma lucidum strain. In one embodiment, the mycelium is derived from a Pleurotus ostreatus strain. In one embodiment, the mycelium is derived from a Fomes fomentarius_strain.

In one embodiment, mycelium is treated with a crosslinking agent in the preparation process. In one embodiment, the crosslinking agent is a tricarboxylic acid. In one embodiment, the crosslinking agent is a tricarboxylic acid selected from citric acid or succinid acid. In one embodiment, the crosslinking agent is a dicarboxylic acid. In one embodiment, the crosslinking agent is a dicarboxylic acid selected from galactaric acid or suberic acid. In one embodiment, the cross-linking agent is a tannin. In one embodiment, an enzyme, such as oxidase or an oxidoreductase is used to crosslink tannins, lignin or vanillin into the mycelium material. In one embodiment, laccase or tyrosinase is used to crosslink tannins, lignin or vanillin into the mycelium material.

In one embodiment, mycelium is treated with mixing/agitation in and/or during the preparation process. In one embodiment, the mycelium is mixed/agitated for a period of time from 5 min to 12 hours. In one embodiment, the mycelium is mixed for a period of time from 5 min to 1 hour. In one embodiment, the mycelium is mixed for about 30 min.

A plasticizer or softener can be used together with the crosslinking agent and/or agitation. The plasticizer can be selected from glycols, sugar alcohols, epoxy esters, ester plasticizers, glycerol esters, phosphate esters, terephtalates, leather conditioners, acetylated monoglycerides, alkyl citrates, epoxidized vegetable oils, methyl ricinoleate, other common polymer plasticizers or any mixture thereof. In one embodiment, the plasticizer is a glycol, such as propylene glycol, polyethylene glycol or a mixture thereof. In one embodiment, the plasticizer is a sugar alcohol, such as glycerol, sorbitol, xylitol or a mixture thereof. In one embodiment, the plasticizer is a leather conditioner, such as a wax, an oil or a mixture thereof. In one embodiment, the plasticizer is a mixture of a sugar alcohol and a glycol.

The present invention relates to mycelium based non-woven material produced in a stirred submerged liquid cultivation. In one embodiment, the invention relates to mycelium based non-woven material produced in a stirred bioreactor. In one embodiment, the mycelium based non-woven material is produced by any of the methods of the present invention. In one embodiment, the mycelium is derived from a Trichoderma reesei strain. In one embodiment, the mycelium is derived from a Ganoderma lucidum strain. In one embodiment, the mycelium is derived from a Pleurotus ostreatus strain. In one embodiment, the mycelium is derived from a Fomes fomentarius_strain.

The non-woven materials of the present invention look like leather and have a leathery feel. They can be used in several applications instead of leather, for example. The mycelium-based non-woven material of the present invention differs from the one produced by a solid-state process in its structure and/or texture. The length of the filaments, the network formed from the filaments and the branching in the filamentous structure of the mycelium produced in the stirred bioreactor differs from the foam-like structure of the material produced in the solid-state process. For example, the filaments grown in a solid-state process are longer and less branched.

The following examples are given to illustrate the invention without, however, restricting the invention thereto.

### EXAMPLE 1

### Production of T. reesei Biomass

Filamentous fungus Trichoderma reesei strain was cultivated in a liquid media containing 15 g/L KH₂PO₄, 5 g/L (NH₄)₂SO₄, 1 ml/L Trace elements and 2 g/L Peptone. The pH was adjusted to 4.8 with KOH and the bottle was filled into the volume of 900 mL with DDIW. The media was autoclaved at 120° C. for 15 min. After that 100 mL of sterile 40% glucose, 2.4 ml of sterile 1M MgSO₄ and 4.1 ml sterile 1M CaCl₂ were added.

T. reesei strain was grown in sterile 2 L Erlenmeyer flasks containing 300 mL of culture media. The flasks were inoculated with 0.001% spore suspension (10⁹ spores/mL) and incubated under 200 rpm shaking at +28° C. for 5 days. The obtained dry weight of the biomass was approximately 6 g/L.

### EXAMPLE 2

### Production of Ganoderma lucidum, Pleurotus ostreatus, and Fomes fomentarius Biomass

Ganoderma lucidum, Pleurotus ostreatus and Fomes fomentarius strains were cultivated on PDA (Potato Dextrose Agar) plates in dark at +28° C. for 10 days. For pre-cultures and production cultivations a Standard Nutritional Liquid (SNL) media (30 g/L D-glucose monohydrate, 4.5 g/L L-asparagine monohydrate, 3 g/L yeast extract, 1.5 g/L KH₂PO₄, 0.5 g/L MgSO₄.H₂O and 1 mL/L trace elements solution) were used. The pH was adjusted to 6.0 with KOH and the media was autoclaved at 121° C. for 20 min.

Pre-cultures were grown in 100 mL Erlenmeyer flasks containing 50 mL of SNL media. For that, three pieces (0.5 cm×0.5 cm) of actively growing mycelium from PDA plates were transferred into 50 mL Falcon tubes and 5 mL of SNL media was added in each tube. The mycelium was homogenized for 15 s by using disperser (Ultra-Turrax). This homogenized mycelium was used as an inoculant (10% v/v) for the pre-cultures. The pre-cultures were incubated under 150 rpm shaking at +28° C. for 8 days.

The production cultivations of G. lucidum, P. ostreatus and F. fomentarius strains were performed in 2 L Erlenmeyer flasks containing 300 mL of SNL media. For that, the pre-cultures were first homogenized (15 s). The flasks were inoculated with 10% (v/v) of homogenized pre-culture and incubated under 150 rpm shaking at +28° C. for 5 days.

### EXAMPLE 3

### Cross-Linking with Citric Acid

Mycelium cultivated in liquid suspension was filtered through GF/B Glass Microfiber filter (GE Healthcare) and washed with DDIW. The filtered biomass was resuspended in DDIW and dry matter content was adjusted to 2% (w/v).

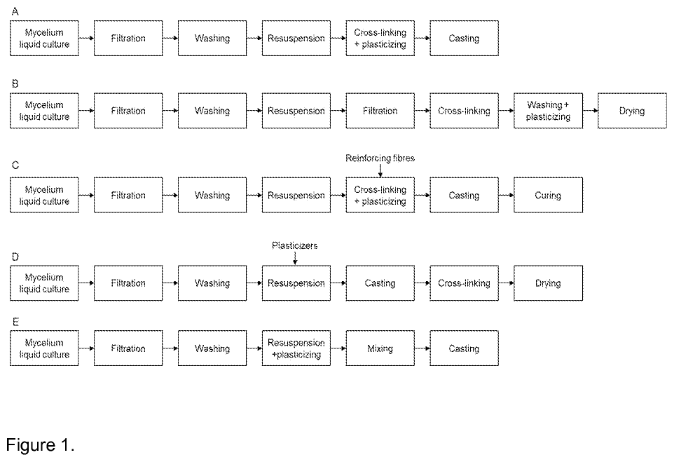

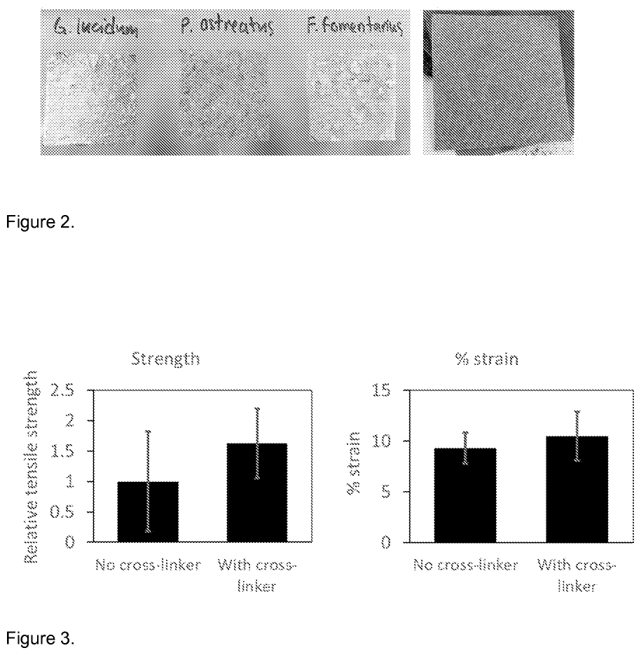

The mycelium suspension was mixed with citric acid and plasticizer. The citric acid and plasticizer contents in the samples were 20% and 20% of the mycelium dry matter content, respectively. The samples were mixed well by using vortex (Vortex-Genie 2, Scientific industries) and the pH was adjusted to 3 with HCl. The samples were dried at +70° C. for 6 h followed by air-drying at room temperature (FIGS. 1A and 2). Tensile measurements showed on increase in tensile strength of the cross-linked samples (FIG. 3).

### EXAMPLE 4

### Cross-Linking with Glutaraldehyde

Mycelium cultivated in liquid suspension was filtered through GF/B Glass Microfiber filter (GE Healthcare) and washed with DDIW. The filtered biomass was resuspended in DDIW and dry matter content was adjusted to 2% (w/v).

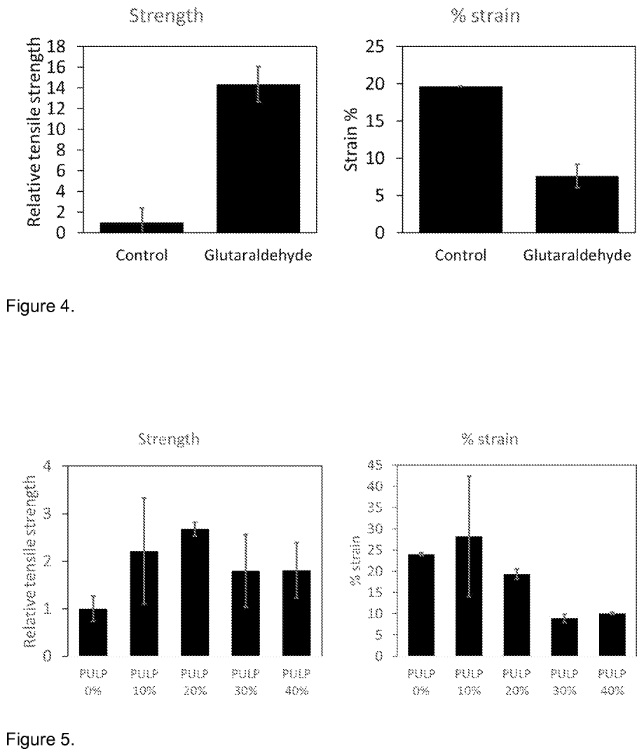

The mycelium suspension was filtered again through a GF/B filter to near dryness and the resulting film was immersed in a 1% glutaraldehyde solution supplemented with 4 mM HCl to lower the pH and incubated over-night. After the reaction the film was rinsed three times thoroughly with DDIW, followed by immersion in 2% glycerol solution and dried by air-drying at room temperature (FIG. 1B). The resulting samples were tested with uniaxial tensile tester. Cross-linked samples showed 14-fold increased ultimate tensile strength and reduced strain values (FIG. 4).

### EXAMPLE 5

### Composite with Fibres

Mycelium cultivated in liquid suspension was filtered through GF/B Glass Microfiber filter (GE Healthcare) and washed with DDIW. The filtered biomass was resuspended in DDIW and dry matter content was adjusted to 2% (w/v).

The samples were prepared by mixing 0, 10, 20, 30, 40 or 100% (on dry matter basis) cellulose pulp from pine with 2% mycelium solution followed by the addition of 20% citric acid and 20% glycerol of total dry matter content. The samples were mixed well with vortex (Vortex-Genie 2, Scientific industries) and dried at +70° C. for 6 h followed by air-drying at room temperature. After that, the samples were placed at +100° C. for 6 min in order to confirm the final curing (FIG. 1C). The resulting samples were tested for mechanical properties using a tensile tester. Increasing fraction of cellulose pulp increased tensile strength up to 2.7-fold at 20% pulp (FIG. 5).

### EXAMPLE 6

### Tannin Treatment

Mycelium cultivated in liquid suspension was filtered through GF/B Glass Microfiber filter (GE Healthcare) and washed with DDIW. The filtered biomass was resuspended in DDIW and dry matter content was adjusted to 2% (w/v).

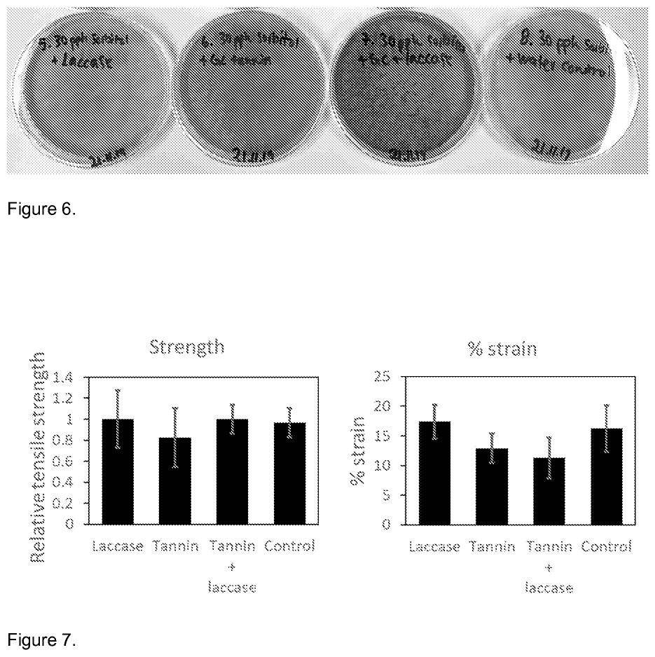

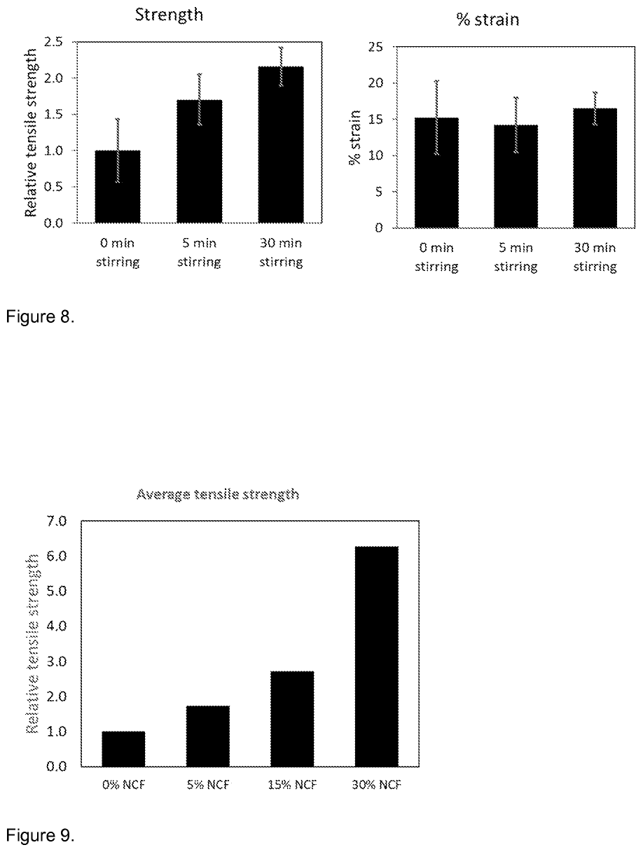

The mycelium suspension was mixed with 30% (of mycelium biomass) of plasticizer followed by drying at 50° C. until almost dry. A solution containing 0.04 g tannin and/or 250 nkat of Trametes hirsuta laccase enzyme was added and the film was dried at 50° C. for 5 h or until dry (FIG. 1D). As a control, only DDIW was added. The resulting non-woven materials showed similar mechanical behavior in tensile tests (FIG. 7). In haptic assessment the leather-like feel was observed to be improved in the tannin containing sample and significantly improved in the sample containing both tannin and laccase enzyme (FIG. 6).

### EXAMPLE 7

### Effect of Stirring

Mycelium cultivated in liquid suspension was filtered through GF/B Glass Microfiber filter (GE Healthcare) and washed with DDIW. The filtered biomass was resuspended in DDIW and dry matter content was adjusted to 2% (w/v). Mycelium suspension with added plasticizer was incubated for 30 min at room temperature and mixed with a magnetic stirrer for 0, 5, or 30 min at about 1000 rpm during the incubation period. The suspensions were then dried at 70° C. for 5 hours or until dry (FIG. 1E). The tensile properties showed increased ultimate tensile strength values with increasing mixing time (FIG. 7). With 30 min mixing the tensile strength more than double as compared to the control sample. The strain values were similar in all samples.

### EXAMPLE 8

### Mechanical Properties

The uniaxial tensile properties were determined with a Universal Tensile Testing machine (Lloyd Instruments) for samples equilibrated to 50% RH and 25° C. and cut to rectangular strips. A 100 N load cell was used with an extension rate of 5 mm/min. Preload was adjusted to 0.1 N. Sample thickness was measured with a L&W Micrometer 51 (Lorentzen & Wettre).

### EXAMPLE 9

### Composite with Nanocellulose Fibrils

Mycelium cultivated in liquid suspension was filtered through GF/B Glass Microfiber filter (GE Healthcare) and washed with DDIW. The filtered biomass was resuspended in DDIW and dry matter content was adjusted to 6% (w/v). The samples were prepared by mixing 0, 5, 15, 30 w-% (of total sample dry matter) nanocellulose fibrils with the mycelium suspension followed by the addition of 20 w-% sorbitol (of total sample dry matter content). The samples were homogenized for 5 min with a high-performance dispersing instrument, air-bubbles were removed by centrifugation under vacuum, poured into a form and dried at +70° C. for 6 h or until dry. The resulting samples were tested for mechanical properties using a tensile tester. Increasing the fraction of nanocellulose fibrils increased tensile strength up to 626% (FIG. 9).

1. A method of making non-woven material from mycelium produced in a stirred submerged liquid culture, wherein the method comprises treating the mycelium suspension with a crosslinking agent and/or agitating the mycelium suspension during the preparation process.

2. The method of claim 1, wherein the method comprises first producing the mycelium in a stirred submerged liquid culture.

3. The method of claim 1, wherein the method comprises treating the mycelium suspension with a crosslinking agent.

4. The method of claim 1, wherein the method comprises treating the mycelium suspension with agitation.

5. The method of claim 3, wherein the cross-linking agent is selected from tricarboxylic acids, dicarboxylic acids, glutaraldehyde or tannins.

6. The method of claim 3, wherein an oxidase or an oxidoreductase is used with the crosslinking agent.

7. The method of claim 3, wherein the crosslinking agent is used with heat-treatment.

8. The method of claim 1, wherein the mycelium is produced by stirred bioreactor cultivation.

9. The method of claim 8, wherein the mycelium is produced by stirred semi-solid-state cultivation.

10. The method of claim 3, wherein the crosslinking agent is separately added to the mycelium.

11. The method of claim 3, wherein the crosslinking agent is produced by the fungus producing the mycelium.

12. The method of claim 4, wherein the agitation is performed for a period of time from 1 min to 12 hours or from 5 min to 1 hour or for about 30 min.

13. The method according to claim 1, wherein a plasticizer is used with the crosslinking agent and/or agitation.

14. The method of claim 13, wherein the plasticizer is selected from the group consisting of glycols, sugar alcohols, epoxy esters, ester plasticizers, glycerol esters, phosphate esters, terephtalates, acetylated monoglycerides, alkyl citrates, epoxidized vegetable oils, methyl ricinoleate and/or and leather conditioners.

15. The method of claim 14, wherein the plasticizer is selected from glycerol, sorbitol, xylitol and polyethylene glycol.

16. The method of claim 1, wherein the mycelium is combined with a polymer, a fiber and/or a colouring agent during or after the cultivation.

17. The method of claim 16, wherein the polymer or the fiber is selected from the group consisting of nanocellulose fibrils, cellulose nanofibrils, nanofibrillated cellulose, cellulose pulp, cellulose derivatives and/or polyvinyl alcohol.

18. (canceled)

19. (canceled)

20. (canceled)

21. (canceled)

22. (canceled)

23. (canceled)

24. (canceled)

25. (canceled)

26. A method of making non-woven material from mycelium produced in a stirred submerged liquid culture, wherein the method comprises the steps of:
a) providing a mycelium pre-culture, and a nutrient and means for stirred submerged liquid cultivation,
b) culturing the mycelium in the stirred submerged liquid cultivation conditions,
c) separating the mycelium from the nutrient media,
d) optionally washing the mycelium with water,
e) optionally adding a crosslinking agent,
f) optionally agitating the mycelium
g) adding a plasticizer to the mycelium suspension in one of the steps b) to f) or to the mycelium after the step h),
h) drying the mycelium, and
i) optionally heat-treating the mycelium.

27. (canceled)

28. The method of claim 26, wherein the method comprises the steps of
a) providing a mycelium pre-culture, and nutrients and means for stirred submerged liquid cultivation,
b) culturing the mycelium in the stirred submerged liquid cultivation conditions,
c) separating the mycelium from the nutrient media,
d) optionally washing the mycelium with water,
e) adding a plasticizer to the mycelium suspension in one of the steps b) to d) or to the mycelium after the step g),
f) agitating the mycelium, and
g) drying the mycelium.

29. The method according to claim 26, wherein the crosslinking agent is selected from tricarboxylic acids, dicarboxylic acids or tannins.

30. The method of claim 26, wherein the method comprises a step of adding a polymer, a fiber and/or a colouring agent to the mycelium.

31. The method of claim 30, wherein the polymer or the fiber is selected from the group consisting of nanocellulose fibrils, cellulose nanofibrils, nanofibrillated cellulose, cellulose pulp, cellulose derivatives and/or polyvinyl alcohol.

32. (canceled)

33. (canceled)
